(19) United States
(12) Patent Application Publication
(10) Pub. No.: US 2010/0007357 A1
(43) Pub. Date: Jan. 14, 2010

(54) Electrical Impedance Tomography Method and Device

(75) Inventors: Habib Ammari, Paris (FR); Eric Bonnetier, Vaulnaveys Le Haut (FR); Yves Capdebscq, Paris (FR); Mickael Tanter, Bagneux (FR); Mathias Fink, Meudon (FR)

Correspondence Address: MILLER, MATTHIAS & HULL, ONE NORTH FRANKLIN STREET, SUITE 2350, CHICAGO, IL, 60606, US

(73) Assignee: Centre National De La Recherche, Paris Cedex 16 (FR)

(21) Appl. No.: 12/443,138

(22) PCT Filed: Sep. 26, 2007
(86) PCT No.: PCT/FR07/52021

§ 371 Date: Mar. 26, 2009

(30) Foreign Application Priority Data
Sep. 28, 2006 (FR) 0608538

(51) Int. Cl. G01R 27/28; G01N 27/02; A61B 5/053; G06F 17/10

(52) U.S. Cl. 324/649; 708/446

(57) ABSTRACT

Electrical impedance tomography method comprising: an electrical measurement step during which pre-determined electrical conditions are imposed on the surface of a medium to be imaged, while generating a mechanical disturbance at predefined points of the medium by locally modifying the impedance of the medium and an electrical parameter is measured at several points on the surface of the medium; and a calculation step during which the electrical impedance is determined at several points in the internal volume of the medium, taking into account the measurements carried out during the disturbance, as a function of a law for modification of the electrical impedance by this disturbance.

The present invention relates to electrical impedance tomography methods and devices.

More particularly, the invention relates to an electrical impedance tomography method for imaging a medium having a certain internal volume delimited by an external surface, this method comprising:
at least one electrical measurement step during which predetermined electrical conditions are imposed on the surface of the medium and at least one electrical parameter is measured at several points on the surface of the medium while generating a mechanical disturbance at predefined points of the medium thus locally modifying the impedance of the medium,
and at least one calculation step during which at least one parameter, linked to the electrical impedance, is determined at several points in the internal volume of the medium.

The document US-A-2003/028092 describes an example of such a method.

All the known electrical impedance tomography methods, including the method described in the abovementioned document, suffer from poor resolution (of the order of a few centimetres for medical uses or of the order of a few tens of metres in the field of geophysics) depending on the depth relative to the surface of the medium to be imaged.

A particular purpose of the present invention is to remedy these drawbacks.

To this end, according to the invention, a method of the kind in question is characterized in that during the calculation step, said parameter, linked to electrical impedance, is determined taking into account the measurements carried out during said disturbance, using a predetermined law for modification of the electrical impedance by said disturbance.

Thanks to these arrangements, it is possible to considerably increase the precision and rapidity of implementation of the method according to the invention, due to the fact that the abovementioned disturbance carries out a local “marking” of the medium. In particular, it is possible to obtain millimetric precision, even to fairly great depths relative to the external surface of the medium, for a very moderate implementation cost.

In various embodiments of the method according to the invention, it is also possible optionally to resort to one and/or other of the following arrangements:
the electrical conditions imposed include at least one current imposed at least one point on the surface of the medium, and said measured electrical parameter is an electrical potential (it is of course possible to impose a potential and to measure currents);
said parameter linked to the electrical impedance is the conductivity;
the mechanical disturbance is a wave focussed on at least one point in the medium;
the wave is an acoustic wave;
the acoustic wave is an ultrasonic wave (generated for example by a set of piezoelectric transducers);
the acoustic wave corresponds to an amplitude-modulated signal at a modulation frequency suitable for generating an ultrasound radiation force resulting in a local displacement of the medium;
the wave is an elastic wave (a mixture of compression waves and shear waves, generated for example by a set of mechanical vibrators arranged on the surface of the soil);
the wave corresponds to an encoded signal;
during the calculation step, the following equation is solved:

$$γ ( z ) = \frac{{E}^{k} ( z )}{A ∇ {u}^{k} ( z ) \cdot ∇ {u}^{k} ( z )} \qquad ( 8 )$$

for any point z of the medium to be imaged, where:

k is an index denoting a set of at least one electric current i, applied to the surface of the medium, 1 being an index denoting each electric current in this set,
E^{k}(z) is an energy corresponding to the disturbance produced by the wave during the application of the set of electric currents jᵢ^{k},
u^{k}(z) is the electrical potential at the point z of the medium,
and A is a matrix representative of the shape of a focal spot produced by the wave about the point on which it is focussed;
the focal spot is spherical and A is the identity matrix;
during the calculation step, said energy is determined as a function of said law for modification of the electrical impedance by said disturbance;
said energy is calculated by the formula:

$${E}^{k} ( z ) = {\sum }_{i} {D}_{i}^{k} ( z ) {j}_{i}^{k} , \qquad ( 6 )$$

where:
z denotes a point situated in the medium,
Dᵢ^{k}(z) is a value representative of the electrical disturbance measured at an index point i on the surface of the medium and generated by the wave during the application of the set of electric currents jᵢ^{k} at the points i;
   Dᵢ^{k}(z) corresponds to the following formula:

Dᵢ^{k}(z)=γA∇u^{k}(z)·∇G(z,i), (3)

where γ is the conductivity and G(z,i) the Neumann function of the medium;
the Dᵢ^{k}(z) values are calculated from the measurements carried out, using waves corresponding to different signals Sₗ(t), l being an index comprised between 1 and L;
   L is equal to 2 and two signals are used, S₁(t)=S₁.s(t) and S₂.s(t)=S2. s (t) of respective amplitudes S₁ and S₂, the Dᵢ^{k}(z) values being calculated, when the shape of the focal spot is a disc or a sphere, by the formula:

$${D}_{i}^{k} ( z ) = \frac{( {u}_{i}^{k , 1} ( z ) - {u}_{i}^{k} ) ( {u}_{i}^{k , 2} ( z ) - {u}_{i}^{k} ) ( {S}_{1} - {S}_{2} )}{d ( {S}_{2} {u}_{i}^{k , 1} ( z ) - {S}_{2} {u}_{i}^{k} - {S}_{1} {u}_{i}^{k , 2} ( z ) + {S}_{1} {u}_{i}^{k} ) \vert V \vert} . \qquad ( 4 )$$

where:
d is either equal to 2 for two-dimensional imaging, or equal to 3 for three-dimensional imaging,
   |V| is either the area of the focal spot for two-dimensional imaging, or the volume of the focal spot for three-dimensional imaging;
   the wave is an ultrasonic wave, L is equal to 2 and two signals are used, S1(t)=S1.s(t) and S2(t)=S2.s(t) of respective amplitudes S1 and S2, s(t) being an amplitude-modulated signal at a modulation frequency suitable for generating an ultrasound radiation force resulting in a local displacement of the medium, the Dᵢ^{k}(z) values being calculated, when the shape of the focal spot is a disc or a sphere, by the formula:

$${D}_{i}^{k} ( z ) = \frac{( {u}_{i}^{k , 1} ( z ) - {u}_{i}^{k} ) ( {u}_{i}^{k , 2} ( z ) - {u}_{i}^{k} ) ( {S}_{1}^{2} - {S}_{2}^{2} )}{d ( {S}_{2}^{2} {u}_{i}^{k , 1} ( z ) - {S}_{2}^{2} {u}_{i}^{k} - {S}_{1}^{2} {u}_{i}^{k , 2} ( z ) + {S}_{1}^{2} {u}_{i}^{k} ) \vert V \vert} . \qquad ( {4}^{′} )$$

where:
d is either equal to 2 for two-dimensional imaging, or equal to 3 for three-dimensional imaging,
   |V| is either the area of the focal spot for two-dimensional imaging, or the volume of the focal spot for three-dimensional imaging;
   during the calculation step, starting with an assumed conductivity γ the following sub-steps are repeated:
      a) the following equation is solved numerically:

$$\{ \begin{matrix}div ( γ ∇ {u}^{k} ) = 0 & \begin{matrix}at any point z of the medium on the \\ external surface\end{matrix} \\ γ ∇ {u}^{k} \cdot \vec{n} = {j}_{k} & \end{matrix} \qquad ( 9 )$$

γ being the previously estimated value of the conductivity (initially, γ is therefore the abovementioned assumed value),
b) an estimated error e^{k} in the conductivity is calculated,
c) the following equation is solved:

$$\{ \begin{matrix}div ( γ ∇ {v}^{k} ) = - div ( {e}^{k} ∇ {u}^{k} ) & \begin{matrix}at any point z of the medium on \\ the external surface\end{matrix} \\ γ ∇ {u}^{k} \cdot \vec{n} = 0 & \end{matrix} \qquad ( 11 )$$

where v^{k} is the solution of the equation (9) and uₖ the solution of the equation (11),
d) the conductivity is updated by the formula:

$${γ}_{k} = - γ ( 2 \frac{{A}^{\frac{1}{2}} ∇ {u}^{k} \cdot ∇ {u}^{k}}{{\vert {A}^{\frac{1}{2}} ∇ {u}^{k} \vert}^{2}} ) + {e}^{k} , \qquad ( 12 )$$

where v^{k} is the solution of the equation (11) and uₖ the solution of the equation (9),
and γₖ is used as a new conductivity value γ with another set of currents jᵢ^{k}, generating lines of current not parallel to those generated by the set of currents jᵢ^{k}, in at least one zone of the medium,
sub-steps a) to d) being reiterated until a stop criterion is satisfied;
during sub-step b), an estimated error e^{k} in the conductivity is calculated by the formula:

e^{k}=E^{k}/A∇u^{k}·∇u^{k}−γ; (10)
during sub-step b), an estimated error e^{k} in the conductivity is calculated by the formula:

$${e}^{h} = \frac{( {E}^{k} / A ∇ {u}^{k} \cdot ∇ {u}^{k} - γ )}{( \vert {E}^{k} / A ∇ {u}^{k} \cdot ∇ {u}^{k} - γ \vert + 1 )} \qquad ( {10}^{′} )$$

during sub-step (d):

for each point z of the medium, it is sought what index k of electric conditions corresponds to the greatest energy

$${\vert {A}^{\frac{1}{2}} ∇ {u}^{k} \vert}^{2}$$

which produces a function k(z),

the conductivity is updated as follows

$${γ}_{k ( z )} = - γ ( 2 \frac{{A}^{\frac{1}{2}} ∇ {u}^{k ( z )} \cdot ∇ {v}^{k ( z )}}{{\vert {A}^{\frac{1}{2}} ∇ {u}^{k ( z )} \vert}^{2}} ) + {e}^{k ( z )} ,$$

and γ_{k(z)} is used as the conductivity value γ;
the medium to be imaged is a biological tissue;
the medium to be imaged is a human organ (for example: breast, liver, brain, or others);
the medium to be imaged is the terrestrial subsoil.

Moreover, a subject of the invention is also a device suitable for the implementation of a method as defined above.

Other characteristics and advantages of the invention will become apparent from the following description of one of its embodiments, given as a non-limitative example, with reference to the attached drawings.

In the drawings:

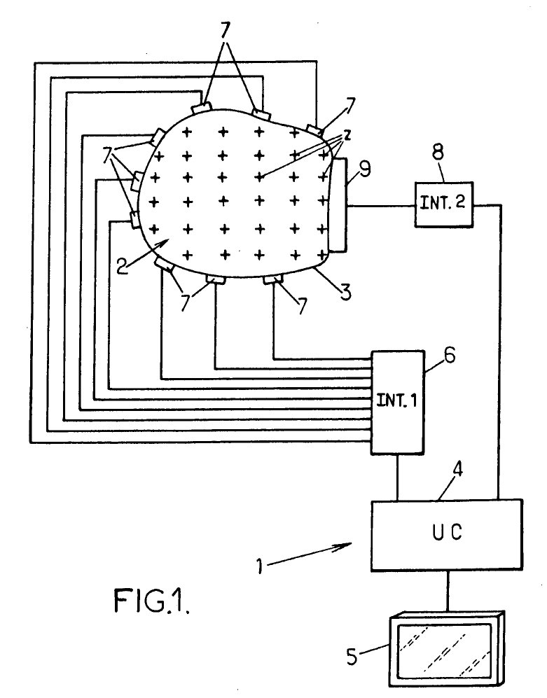

FIG. 1 is a diagrammatic view of an electrical impedance tomography device according to an embodiment of the invention,

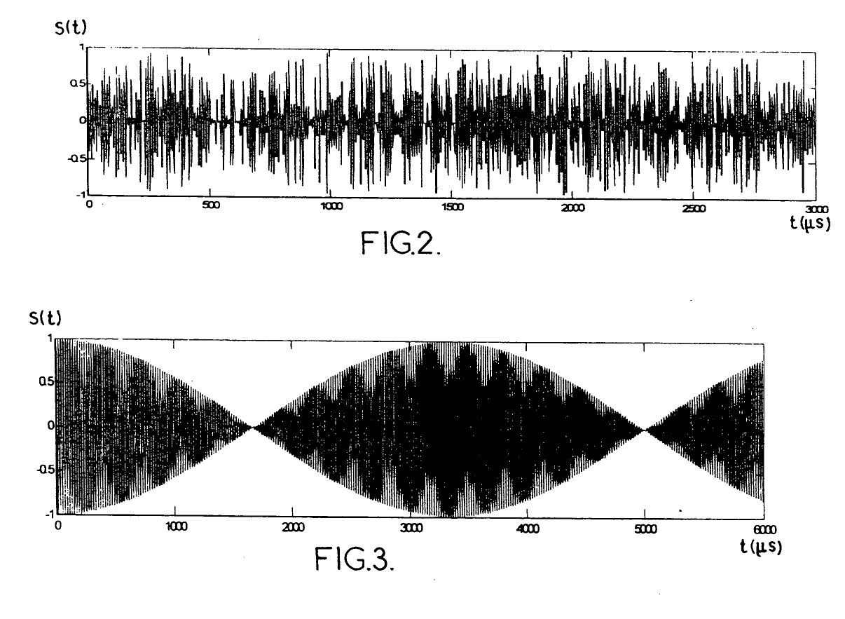

FIG. 2 is a graph representing the signal corresponding to the ultrasonic waves applied by the device of FIG. 1,

and FIG. 3 is a graph similar to FIG. 1, for a variant of the invention.

In the different figures, the same references denote identical or similar elements.

FIG. 1 shows an electrical impedance tomography device 1, used for imaging a medium (for example a biological medium, for example a human body part such as a breast, a liver, a brain or any other organ) which has a certain internal volume 2 delimited by an external surface 3.

The device 1 comprises a central unit 4 (UC) such as a computer or similar, which can be connected to various peripherals such as a screen 5 and other input and output interfaces (not shown).

The central unit 4 is connected to an electrical measurement interface 6 (INT. 1) such as those conventionally used in electrical impedance tomography, connected to a plurality of electrodes 7 arranged on the surface 3 of the medium to be imaged. The electrodes 7 number I (non-zero natural number) and are each denoted by an index i comprised between 1 and I.

The electrical measurement interface 6 is suitable for imposing predetermined electrical conditions on certain of the electrodes 7 and for measuring at least one electrical parameter at the level of all or some of the electrodes 7. For example, the electrical measurement interface 6 can be suitable for imposing at least one predetermined current j at the level of one of the electrodes 7 and for measuring voltages uᵢ at the level of all the electrodes 7. More particularly, the central unit 4 is suitable for controlling the electrical measurement interface 6 such that it successively imposes several currents jₗ, . . . Jₖ, . . . jₖ (k being an index denoting each measurement and K a non-zero natural number denoting the total number of measurements with different currents: each current of index k can differ from the other currents by the electrode 7 to which it is applied and/or by the signal to which it corresponds) and for measuring the voltages Uᵢ for each current jₖ.

Alternatively, the electrical measurement interface 6 could be suitable for imposing one or more voltages and measuring currents at the level of the electrodes 7.

The currents and voltages in question can for example be alternating, for example with a frequency of the order of one kHz.

Moreover, the central unit 4 is connected to a signal processing interface 8 (INT. 2) which controls an array 9 of ultrasonic piezo-electric transducers (for example a straight strip of transducers) applied to the surface 3 of the medium to be imaged. It will be noted that the array 9 of transducers can also be a two-dimensional array and/or be mounted on a mobile support allowing variation of the position and/or the orientation of the array.

The signal processing interface 8 is suitable for making the array 9 of transducers generate ultrasonic waves successively focussed on predetermined points z (z in number) situated in the medium to be imaged, at least certain of these points z being situated in the internal volume 2 (the other being optionally on the surface 3). The ultrasonic waves in question can be for example of a frequency comprised between 0.5 and 15 MHz, in particular of the order of one MHz.

When it is desired to image the internal volume 2 of the medium, the central unit 4 successively applies, by the electrical measurement interface 6, currents jₖ to all or some of the electrodes 7.

For each current jₖ, the electrical measurement interface 6 measures the voltage (uᵢ^{k})_{1≦i≦I} of each electrode 7 of index i, in the absence of acoustic ultrasonic waves in the medium to be imaged.

Then the central unit 4 causes the emission, by the signal processing interface 8 and the array of transducers 9, of ultrasonic waves focussed successively on the abovementioned predetermined points z, in order to generate mechanical disturbances of the medium localized at each point z, resulting in localized disturbances of the electrical impedance (and in particular of the conductivity) of the medium.

The ultrasonic wave is focussed on each point z for a few hundred periods of the ultrasonic wave.

The ultrasonic wave in question can be a wave which is not low-frequency modulated, which induces a local and infinitesimal variation in volume in the focal zone of the ultrasonic beam. The frequency at which this vibration is produced is the ultrasound excitation frequency. Local disturbances of the electrical impedance are then produced, having the same frequency as the ultrasound signal. In order to increase the signal-to-noise ratio necessary to detect the influence of the ultrasonic beam on the electric signals, it is possible for example to emit an encoded ultrasound signal S(t), such as for example that shown in FIG. 2.

As an encoded signal, it is possible to use for example a “chirp” function S(t)=sin(2π(f0+Δf.t)t) where f0 is a frequency and Δf a frequency bandwidth. As a variant, it is also possible to use as coding a predetermined embodiment of white noise.

More generally, it is possible to successively cause the emission of ultrasonic waves corresponding to signals Sₗ(t), . . . Sₗ(t), . . . S_{L}(t), l being an exponent comprised between 1 and L, L being a non-zero natural number (L=1 if only one single signal S(t) is used). In this case, the waves corresponding to each signal Sₗ can for example be focussed successively on the different points z, before emitting and focussing the waves corresponding to the signal Sₗ₊₁. It will be noted that the signals Sₗ(t) can optionally differ from each other only by their amplitude.

While the current jₖ and the signal Sₗ are applied and the ultrasonic wave corresponding to the signal Sₗ is focussed on a given point z, the voltages (uᵢ^{k, l, z})_{1≦i≦l} are measured at the I electrodes 7 of indices i. If the signal S₁(t) is encoded as indicated above, the sensitivity of the measurement can be improved by deconvolution of the electrical signal (uᵢ^{k, l, z})_{1≦i≦I} by the code applied to the signal Sₗ(t).

In total, the central unit stores the I.K voltage measurements (uᵢ^{k})_{1≦i≦I, 1≦k≦K} carried out without focussing of acoustic waves and the I.K.L.Z voltage measurements (uᵢ^{k,l,z})_{1≦i≦I, 1≦k≦K, 1≦l≦L} carried out with focussing of acoustic waves.

On the basis of these measurements, a reconstruction calculation is carried out, which involves finding the conductivity γ(z) at any point z of the medium to be imaged.

This reconstruction corresponds to the following problem:

$$\{ \begin{matrix}div ( γ ( z ) ∇ {u}^{k} ( z ) ) = 0 & \begin{matrix}at any point z of the medium on the \\ external surface\end{matrix} \\ γ ( z ) ∇ {u}^{k} \cdot \vec{n} = {j}_{k} & \end{matrix} \qquad ( 1 )$$

where u^{k} is the electric voltage (potential) on the external surface 3, u^{k}(z) is the electric voltage at the point z in the internal volume 2 and \vec{n} is the normal to the external surface 3 of the medium.

In order to solve this mathematical problem, it is possible optionally to use a standard method which involves testing different conductivities by minimizing the difference from the measured data, for example by the method of least squares.

More advantageously, it is possible to use the method described below, which has proved particularly advantageous, precise and robust.

This method is based on the fact that, as taught by the works of H. Ammari and H. Kang (“Reconstruction of Small Inhomogeneities from Boundary Measurements, Lecture notes in mathemetics”, volume 1846, Springer Verlag, Berlin, 2004), the electrical disturbance due to a change in local conductivity at a point z of the medium to be imaged is given primarily by the formula:

uᵢ^{k,l,z}−uᵢ^{k}=(γₚ−γ)M∇u^{k}(z)·∇G(z,i), (2)

where:
uᵢ^{k} is the potential at the point i and uᵢ^{k,l,z} is the potential due to the ultrasonic disturbance which corresponds to the signal 1, focussed at the point z,
γₚ is the conductivity disturbed locally by the ultrasounds,
M is a geometric factor, the polarization tensor, which depends on γₚ and on the shape of the focal zone of the ultrasonic wave (for example, the zone in which the amplitude of the ultrasonic wave is greater than half the maximum amplitude)
the function G is the Neumann function of the medium of conductivity γ and therefore unknown.

It is possible to extract, from these electrical disturbances, a matrix D representative of the electrical disturbance at the point z in the presence of the current k:

Dᵢ^{k}(z)=γA∇u^{k}(z)·∇G(z,i) (3)

where A is a known positive definitive matrix dependent only on the shape of the focal zone.

This matrix A is linked to the polarization tensor M by the formula A 1/d (γ/γₚ+(d−1))M, where d is the space dimension (d=2 for two-dimensional imaging and d=3 for three-dimensional imaging).

The shape of the focal zone of the ultrasonic wave being known, this matrix A is known. For example, for a circular or spherical focal zone, A is equal to the identity matrix Id.

The matrix D can be calculated from the measurements carried out, using only the differences in amplitudes of the ultrasonic waves corresponding to different signals Sₗ(t).

For example, starting from two signals Sₗ(t)=Sₗ.s(t) and S₂(t)=S₂.s(t) of respective amplitudes Sₗ and S2, the matrix in question can be calculated, when the shape of the focal spot is a disc or a sphere, by the formula:

$${D}_{i}^{k} ( z ) = \frac{( {u}_{i}^{k , 1} ( z ) - {u}_{i}^{k} ) ( {u}_{i}^{k , 2} ( z ) - {u}_{i}^{k} ) ( {S}_{1} - {S}_{2} )}{d ( {S}_{2} {u}_{i}^{k , 1} ( z ) - {S}_{2} {u}_{i}^{k} - {S}_{1} {u}_{i}^{k , 2} ( z ) + {S}_{1} {u}_{i}^{k} ) \vert V \vert} . \qquad ( 4 )$$

where:
d is the space dimension (2 for two-dimensional imaging and 3 for three-dimensional imaging,
   |V| is the area (for two-dimensional imaging) or the volume (for three-dimensional imaging) of the ultrasound focal zone, and Sₗ and S₂ the amplitudes of the ultrasonic waves.

This matrix D makes it possible to calculate the electrical energy E^{k}(z) equivalent to the acoustic disturbances at the points z, for each current k.

This electrical energy is defined by the formula:

E^{k}(z)=γ(z)A∇u^{k}(z)·∇u^{k}(z) (5)

and calculated in practice by the formula:

$$\begin{matrix}{E}^{k} ( z ) = {\int }_{Ω}^{ } {D}^{k} ( z , y ) {j}^{k} ( y ) d y \\ = {\sum }_{i} {D}_{i}^{k} ( z ) {j}_{i}^{k}\end{matrix} \qquad ( 6 )$$

It is also possible to use other quadrature formulae, for example when the currents are not measured at the level of the electrodes i but at points distinct from the external surface 3 of the medium to be imaged.

By carrying formula (6) into formula (1), the mathematical problem to be solved can be written as follows:

$$\{ \begin{matrix}div ( \frac{{E}^{k}}{A ∇ {u}^{k} \cdot ∇ {u}^{k}} ∇ {u}^{k} ) = 0 & \begin{matrix}\begin{matrix}at any point z of the internal \\ volume 2 of the medium to be \end{matrix} \\ imaged on the external surface \\ 3 of the medium to be imaged\end{matrix} \\ γ ∇ {u}^{k} \cdot \vec{n} = {j}_{k} & \end{matrix} \qquad ( 7 )$$

It will be noted that, if appropriate, the conductivity γ on the external surface 3 of the medium to be imaged can be determined by means of independent measurements, without resorting to this method.

This non-linear equation which contains no unknown coefficient is solved, giving u^{k}(z) at any point z of the medium to be imaged. This produces

$$γ ( z ) = \frac{{E}^{k} ( z )}{A ∇ {u}^{k} ( z ) \cdot ∇ {u}^{k} ( z )} \qquad ( 8 )$$

for any point z of the medium to be imaged.

To solve this non-linear equation, it is possible to use the following algorithm:
1/starting from a supposed conductivity γ for example γ=1 at any point of the medium,
2/the following steps are repeated:
   a) with a standard linear solver for a current jk the following problem is solved numerically

$$\{ \begin{matrix}div ( γ ∇ {u}^{k} ) = 0 & \begin{matrix}at any point z of the medium on the \\ external surface\end{matrix} \\ γ ∇ {u}^{k} \cdot \vec{n} = {j}_{k} & \end{matrix} \qquad ( 9 )$$

b) the error is calculated

c^{k}=E^{k}/A∇u^{k}·∇u^{k}−γ, (10)

E^{k} being the energy calculated by the formula (6);
c) the following problem is solved

$$\{ \begin{matrix}div ( γ ∇ {v}^{k} ) = - div ( {e}^{k} ∇ {u}^{k} ) & \begin{matrix}at any point z of the medium on \\ the external surface\end{matrix} \\ γ ∇ {u}^{k} \cdot \vec{n} = 0 & \end{matrix} \qquad ( 11 )$$

d) the conductivity is updated by the formula

$${γ}_{k} = - γ ( 2 \frac{{A}^{\frac{1}{2}} ∇ {u}^{k} \cdot ∇ {v}^{k}}{{\vert {A}^{\frac{1}{2}} ∇ {u}^{k} \vert}^{2}} ) + {e}^{k} , \qquad ( 12 )$$

where v^{k} is an electrical potential, solution of the equation (11), and uₖ an electrical potential, solution of the equation (9),
and γₖ, given by the formula (12), is used as a conductivity value γ in the following iteration, for an appropriate set of currents jᵢ^{k} (generating current lines not parallel to those generated by jᵢ^{k}, at least in certain zones of the medium to be imaged).

Steps a) to d) are reiterated until a stop criterion is satisfied, for example:
when a standard error e becomes small,
or when a standard of ∇ᵤ^{k} becomes small.

In practice, approximately ten iterations of steps a) to d) are sufficient for convergence.

As a variant, it is possible to use ultrasonic waves corresponding to relatively low-frequency modulated signals S₁(t), for example with a modulation frequency of a few hundred Hz, as shown in FIG. 3. In this case, the ultrasonic beam induces a localized force in the focal zone which pushes the medium. This force, known as the ultrasound radiation force, causes a local displacement of the medium in which time-based variations are linked not to the ultrasonic frequency, but to the low-frequency envelope of the ultrasound signal. During the use of this technique No. 2, the ultrasound radiation force can also be encoded in time, by modulating in time the amplitude of the ultrasound boost signal.

In this case, it is possible to use the same method as that described above, preferably by replacing formula (4) by the following formula (4′), when the shape of the focal spot is a disc or a sphere:

$${D}_{i}^{k} ( z ) = \frac{( {u}_{i}^{k , 1} ( z ) - {u}_{i}^{k} ) ( {u}_{i}^{k , 2} ( z ) - {u}_{i}^{k} ) ( {S}_{1}^{2} - {S}_{2}^{2} )}{d ( {S}_{2}^{2} {u}_{i}^{k , 1} ( z ) - {S}_{2}^{2} {u}_{i}^{k} - {S}_{1}^{2} {u}_{i}^{k , 2} ( z ) + {S}_{1}^{2} {u}_{i}^{k} ) \vert V \vert} . \qquad ( {4}^{′} )$$

Moreover, in order to make the algorithm more stable and robust, it is possible to replace the error formula (10) by

$${e}^{k} = \frac{( {E}^{k} / A ∇ {u}^{k} \cdot ∇ {u}^{k} - γ )}{( \vert {E}^{k} / A ∇ {u}^{k} \cdot ∇ {u}^{k} - γ \vert + 1 )} \qquad ( {10}^{′} )$$

In this case, the convergence is slower, but very strong contrasts in the materials can be detected without instability.

Moreover, it is also possible to use the data of several currents simultaneously in the above method.

To this end, during step (d):
for each point z of the field it is sought what current k corresponds to the greatest energy

$${\vert {A}^{\frac{1}{2}} ∇ {u}^{k} \vert}^{2}$$

which gives a function k(z),
for each current k(z) the conductivity is updated as follows:

$${γ}_{k ( z )} = - γ ( 2 \frac{{A}^{\frac{1}{2}} ∇ {u}^{k ( z )} \cdot ∇ {v}^{k ( z )}}{{\vert {A}^{\frac{1}{2}} ∇ {u}^{k ( z )} \vert}^{2}} ) + {e}^{k ( z )} ,$$

γ being the previous conductivity value,
and γ_{k(z)} is used as a conductivity value γ in the following iteration.

It will be noted that the method according to the invention can also be used in geophysics. In this case, the medium to be imaged is terrestrial soil and the ultrasonic waves are replaced by elastic waves, in particular low frequency compression or shear waves (for example, from 5 Hz to 5000 Hz). The abovementioned piezo-electric transducers are then replaced by a set of mechanical vibrators or actuators arranged on the surface of the soil.

1. An electrical impedance tomography method for imaging a medium having a certain internal volume delimited by an external surface, this method comprising:
at least one electrical measurement step during which:
predetermined electrical conditions are imposed on the surface of the medium by an electrical measurement apparatus controlled by a central unit, said electrical measurement apparatus having electrodes on the surface of the medium;
and at least one electrical parameter is measured by said electrical measurement apparatus at several points on the surface of the medium while generating a mechanical disturbance at predefined points of the medium by a mechanical disturbance generating apparatus controlled by said central unit, thus locally modifying the impedance of the medium,
and at least one calculation step during which at least one parameter, linked to the electrical impedance, is determined at several points in the internal volume of the medium, wherein during the calculation step, said parameter linked to electrical impedance is determined taking into account the measurements carried out during said disturbance, using a predetermined law for modification of the electrical impedance by said disturbance.

2. The method according to claim 1, in which the electric conditions imposed include at least one current imposed at least one point on the surface of the medium, and said measured electrical parameter is an electrical potential.

3. The method according to claim 1, in which said parameter linked to the electrical impedance is the conductivity.

4. The method according to claim 1, in which the mechanical disturbance is a wave focussed on at least one point of the medium.

5. The method according to claim 4, in which the wave is an acoustic wave.

6. The method according to claim 5, in which the acoustic wave is an ultrasonic wave.

7. The method according to claim 6, in which the wave corresponds to an amplitude-modulated signal at a modulation frequency suitable for generating an ultrasound radiation force resulting in a local displacement of the medium.

8. The method according to claim 4, in which the wave is an elastic wave.

9. The method according to, claim 4, in which the wave corresponds to an encoded signal.

10. The method according to claim 4, in which, during the calculation step, the following equation is solved:
$$γ ( z ) = \frac{{E}^{k} ( z )}{A ∇ {u}^{k} ( z ) \cdot ∇ {u}^{k} ( z )} \qquad ( 8 )$$
for any point z of the medium to be imaged,
where:
k is an index denoting a set of at least one electric current jᵢ^{k} at the surface of the medium, i being an index denoting each current of this set,
E^{k}(z) is an energy corresponding to the disturbance produced by the wave during the application of the set of electric currents jᵢ^{k} at the surface of the medium,
u^{k}(z) is the electrical potential at the point z of the medium,
and A is a matrix representative of the shape of a focal spot produced by the wave about the point on which it is focussed.

11. The method according to claim 10, in which the focal spot is spherical and A is the identity matrix.

12. The method according to claim 10, in which during the calculation step, said energy is determined as a function of said law for modification of the electrical impedance by said disturbance.

13. The method according to claim 10, in which said energy is calculated by the formula:
$${E}^{k} ( z ) = {\sum }_{i} {D}_{i}^{k} ( z ) {j}_{i}^{k} , \qquad ( 6 )$$
where:
z denotes a point situated in the medium,
Dᵢ^{k}(z) is a value representative of the electrical disturbance measured at an index point i on the surface (3) of the medium and generated by the wave during the application of the set of electric currents jᵢ^{k} at the points i.

14. The method according to claim 13, in which Dᵢ^{k}(z) corresponds to the following formula:
Dᵢ^{k}(z)=γA∇u^{k}(z)·∇G(z,i), (3)
where γ is the conductivity and G(z,i) the Neumann function of the medium.

15. The method according to claim 13, in which the Dᵢ^{k}(z) values are calculated from the measurements carried out, using waves corresponding to different signals Sₗ(t), l being an index comprised between l and L.

16. The method according to claim 15, in which L is equal to 2 and two signals are used, Sₗ(t)=S_{l.s}(t) and S₂(t)=S₂.s(t) of respective amplitudes Sₗ and S₂, the Dᵢ^{k}(z) values being calculated, when the shape of the focal spot is a disc or a sphere, by the formula:
$${D}_{i}^{k} ( z ) = \frac{( {u}_{i}^{k , 1} ( z ) - {u}_{i}^{k} ) ( {u}_{i}^{k , 2} ( z ) - {u}_{i}^{k} ) ( {S}_{1} - {S}_{2} )}{d ( {S}_{2} {u}_{i}^{k , 1} ( z ) - {S}_{2} {u}_{i}^{k} - {S}_{1} {u}_{i}^{k , 2} ( z ) + {S}_{1} {u}_{i}^{k} ) \vert V \vert} . \qquad ( 4 )$$
where:
d is either equal to 2 for two-dimensional imaging, or equal to 3 for three-dimensional imaging,

|V| is either the area of the focal spot for two-dimensional imaging, or the volume of the focal spot for three-dimensional imaging.

17. The method according to claim 15, in which the wave is an ultrasonic wave, L is equal to 2 and two signals are used, Sₗ(t)=Sₗ.s(t) and S₂(t)=S₂.s(t) of respective amplitudes Sₗ and S₂, s(t) being an amplitude-modulated signal at a modulation frequency suitable for generating an ultrasound radiation force resulting in a local displacement of the medium, the Dᵢ^{k}(z) values being calculated, when the shape of the focal spot is a disc or a sphere, by the formula:
$${D}_{i}^{k} ( z ) = \frac{( {u}_{i}^{k , 1} ( z ) - {u}_{i}^{k} ) ( {u}_{i}^{k , 2} ( z ) - {u}_{i}^{k} ) ( {S}_{1}^{2} - {S}_{2}^{2} )}{d ( {S}_{2}^{2} {u}_{i}^{k , 1} ( z ) - {S}_{2}^{2} {u}_{i}^{k} - {S}_{1}^{2} {u}_{i}^{k , 2} ( z ) + {S}_{1}^{2} {u}_{i}^{k} ) \vert V \vert} . \qquad ( {4}^{′} )$$
where:
d is either equal to 2 for two-dimensional imaging, or equal to 3 for three-dimensional imaging,

|V| is either the area of the focal spot for two-dimensional imaging, or the volume of the focal spot for three-dimensional imaging.

18. The method according to claim 10, in which, during the calculation step, starting from a supposed conductivity γ the following sub-steps are repeated:
a) the following equation is solved numerically:
$$\{ \begin{matrix}div ( γ ∇ {u}^{k} ) = 0 & \begin{matrix}at any point z of the medium on \\ the external surface ,\end{matrix} \\ γ ∇ {u}^{k} \cdot \vec{n} = {j}_{k} & \end{matrix} \qquad ( 9 )$$
γ being a previously estimated conductivity value,
b) an estimated error e^{k} in the conductivity is calculated,
c) the following equation is solved:
$$\{ \begin{matrix}div ( γ ∇ {v}^{k} ) = - div ( {e}^{k} ∇ {u}^{k} ) & \begin{matrix}at any point z of the medium \\ on the external surface ,\end{matrix} \\ γ ∇ {u}^{k} \cdot \vec{n} = 0 & \end{matrix} \qquad ( 11 )$$
d) the conductivity is updated by the formula:
$${γ}_{k} = - γ ( 2 \frac{{A}^{\frac{1}{2}} ∇ {u}^{k} \cdot ∇ {v}^{k}}{{\vert {A}^{\frac{1}{2}} ∇ {u}^{k} \vert}^{2}} ) + {e}^{k} , \qquad ( 12 )$$
where v^{k} is the solution of the equation (11) and uₖ is the solution of the equation (9),
and γₖ is used as a new conductivity value γ, with another set of currents jᵢ^{k} generating current lines not parallel to those generated by the set of currents jᵢ^{k} in at least one zone of the medium, the sub-steps a) to d) being reiterated until a stop criterion is satisfied.

19. The method according to claim 18, in which, during sub-step b), an estimated error e^{k} in the conductivity is calculated by the formula:
e^{k}=E^{k}/A∇u^{k}·∇u^{k}−γ. (10)

20. The method according to claim 18, in which, during sub-step b), an estimated error e^{k} in the conductivity is calculated by the formula:
$${e}^{k} = \frac{( {E}^{k} / A ∇ {u}^{k} \cdot ∇ {u}^{k} - γ )}{( \vert {E}^{k} / A ∇ {u}^{k} \cdot ∇ {u}^{k} - γ \vert + 1 )} . \qquad ( {10}^{′} )$$

21. The method according to claim 18, in which, during sub-step (d):
for each point z of the medium, it is sought what index k of electric conditions corresponds to the greatest energy
$${\vert {A}^{\frac{1}{2}} ∇ {u}^{k} \vert}^{2}$$
which gives a function k(z),
the conductivity is updated as follows:
$${γ}_{k ( z )} = - γ ( 2 \frac{{A}^{\frac{1}{2}} ∇ {u}^{k ( z )} \cdot ∇ {v}^{k ( z )}}{{\vert {A}^{\frac{1}{2}} ∇ {u}^{k ( z )} \vert}^{2}} ) + {e}^{k ( z )} ,$$
and γ_{k(z)} is used as a conductivity value γ.

22. The method according to claim 1, in which the medium to be imaged is a biological tissue.

23. The method according to claim 22, in which the medium to be imaged is a human organ.

24. The method according to claim 1, in which the medium to be imaged is the terrestrial subsoil.

25. An electrical impedance tomography device for imaging a medium having a certain internal volume delimited by an external surface, said device comprising:
a central unit;
an electrical measurement apparatus having electrodes, said electrical measurement apparatus being controlled by said central unit for imposing predetermined electrical conditions on said electrodes on the surface of the medium and for measuring at least one electrical parameter through at least part of said electrodes on the surface of the medium,
a mechanical disturbance generating apparatus controlled by said central unit for generating a mechanical disturbance at predefined points of the medium by locally modifying the impedance of the medium while measuring said electrical parameter:
calculating means for determining at least one parameter, linked to the electrical impedance, at several points in the internal volume of the medium, said calculating means being adapted to determine said parameter taking into account the measurements carried out during said disturbance, using a predetermined law for modification of the electrical impedance by said disturbance.

26. The electrical impedance tomography device as claimed in claim 25, in which the mechanical disturbance generating apparatus is adapted to generate a wave focussed on at least one point of the medium.
